(19) United States
(12) Patent Application Publication
(10) Pub. No.: US 2008/0027262 A1
(43) Pub. Date: Jan. 31, 2008

(54) Method for Producing Alkenses by the Elimination of Water from Alcohols, Using Alkylphosphonic Acid Anhydrides

(76) Inventors: Andreas Meudt, Hofheim (DE); Stefan Scherer, Griesheim (DE); Claudius Boehm, Frankfurt (DE)

Correspondence Address: PROPAT, L.L.C., 425-C SOUTH SHARON AMITY ROAD, CHARLOTTE, NC, 28211-2841, US

(21) Appl. No.: 11/579,937

(22) PCT Filed: Jun. 4, 2005
(86) PCT No.: PCT/EP05/06016

§ 371 Date: Nov. 8, 2006

(30) Foreign Application Priority Data
Jun. 19, 2004 (DE) 10 2004 029 811.4

(51) Int. Cl. C07C 1/24; C07C 11/02; C07C 13/11; C07C 15/46

(52) U.S. Cl. 585/639

(57) ABSTRACT

The invention relates to a method for producing alkenes of formula R¹R²C═CR³R⁴ by the reaction of a) primary alcolhols (R¹R²CHCH₂—OH) or b) secondary alcohols (R¹R²CHCHR³—OH) or c) tertiary alcohols (R¹R²CH—CR³R⁴OH) with cyclic alkylphosphonic acid anhydrides at a temperature ranging between −100 and +120° C., whereby R and/or R¹ and/or R² and/or R³ and/or R⁴ represent H, a linear or branched C₁-C₁₂ alkyl group, or a C₃-C₁₀ cycloalkyl group, alkenyl group or an aryl group or heteroaryl group. Preferably, a 2,4,6-substituted 1,3,5,2,4,6-trioxatriphosphinane-2,4,6-trioxide of formula (I) is used as the cyclic phosphonic acid anhydride, where R′ represents, (independently of one another), allyl, aryl or open-chained or branched C₁-C₁₂ alkyl groups. Optionally the reaction can be carried out in the presence of a tertiary amine base NR⁵₃.

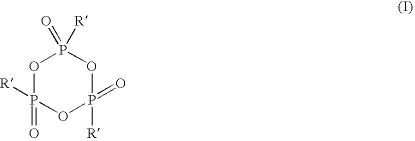

Alkenes are important and extremely versatile intermediates in organic synthesis. This compound class exhibits a high reactivity of the C,C double bond, which enables numerous addition reactions. The significance in modern organic synthesis is restricted only by limitations in the availability of these compound classes. Standard processes for the preparation of alkenes are eliminations of correspondingly functionalized alkanes. Numerous methods such as the elimination of hydrogen halide and halogens, the pyrolysis of esters, ammonium, phosphonium, sulfonium and sulfur compounds, and the elimination of hydrogen are used. It is also possible to dehydrate aliphatic hydroxyl compounds in the vapor phase or in the liquid phase.

In modern organic synthesis, the significance of chemo-, region- and stereoselective reagents is increasing explosively. When, for example, the intention is to convert a specific alcohol functionality to an alkene in a complex molecule with numerous functional groups, numerous methods from those mentioned are ruled out for selectivity reasons.

A selective and preferred method for eliminating alcohols to alkenes is the reaction in the liquid phase. The reagents which are used predominantly for the conversion of alcohols to olefins can be divided into alkaline and acidic dehydrating agents. In addition, other reagents which are not strictly attributable to one of these groups are also used in isolated cases.

There has to date been a lack of a highly selective solution to the problem of the transformations mentioned. Although the known reagents can accomplish the desired transformations, other moieties are often likewise influenced at the same time. In many cases, the drastic conditions required result in epimerization even of far-removed stereocenters.

It is therefore an object of the invention to provide an economically viable process which allows alcohols to be converted to the corresponding alkenes under very mild conditions by elimination of water, and the removal of any conversion products formed from the starting materials used should be performable in a simple manner.

The processes for preparing alkenes already known in the prior art all have serious disadvantages. For instance, when sulfuric acid is used to eliminate water, as well as the carbonization and the risk of contamination of the product with sulfur dioxide, there is also the risk of the formation of ethers. When phosphoric acid is used for dehydration, temperatures of up to 220° C. are usually necessary. Boric acid only finds use occasionally. In the case of the use of primary alcohols, even temperatures of up to 300° C. are necessary. This therefore gives olefin mixtures with nonuniform position of the double bond.

It has been found that, surprisingly, the use of cyclic 2,4,6-substituted 1,3,5,2,4,6-trioxatriphosphinane 2,4,6-trioxides solves all of these problems. This elimination method enables the highly selective conversion of alcohols to the corresponding alkenes, with simultaneous observation of the desired freedom from epimerization and maximum regio- and stereo-selectivity with simultaneously virtually quantitative yields.

The present invention thus relates to a highly selective process for preparing alkenes of the formula (II)
R¹R²C═CR³R⁴ (II)
by reacting
a) primary alcohols (R¹R²CHCH₂—OH) or
b) secondary alcohols R¹R²CHCHR³—OH) or
c) tertiary alcohols (R¹R²CH═CR³R⁴OH)
with cyclic alkylphosphonic anhydrides and optionally an amine base NR₃⁵ at a temperature in the range from −100 to +120° C.,
   where R and/or R¹ and/or R² and/or R³ and/or R⁴ is H, a linear or branched, substituted or unsubstituted C₁-C₁₂-alkyl radical, a C₃-C₁₀-cycloalkyl radical, alkenyl radical or an aryl or heteroaryl radical.

In a preferred inventive embodiment, the cyclic alkylphosphonic anhydride is a 2,4,6-substituted 1,3,5,2,4,6-trioxatriphosphinane 2,4,6-trioxide of the formula (I) 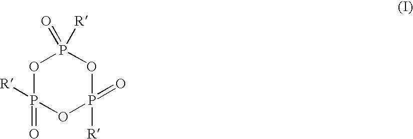
where R′ is independently allyl, aryl or open-chain or branched C₁ to C₁₂-alkyl radicals, in particular C₁-C₈-alkyl radicals.

Particular preference is given to phosphonic anhydrides of the formula (I) in which R¹ is a methyl, ethyl, n-propyl, isopropyl, n-butyl, 2-butyl, isobutyl, pentyl, hexyl, especially an ethyl, propyl and/or butyl radical.

The elimination to alkenes (II) can generally be performed at temperatures in the range from −100 to +120° C.; preference is given to temperatures in the range from −30 to +30° C., lower temperatures generally being correlated with higher selectivities. The reaction time is dependent upon the temperature employed and is generally from 1 to 12 hours, in particular from 3 to 6 hours.

The addition of amines is generally not required, but may prove to be advantageous in the individual case. The amines used are generally amines of the formula (III)
NR⁵₃ (III)
where R⁵ is H, allyl, aryl or open-chain, cyclic or branched C₁- to C₁₂-alkyl radicals, aryloxy, allyloxy or alkoxy having open-chain, cyclic or branched C₁- to C₁₂-alkyl radicals, or a combination of the substituents mentioned.

Particular preference is given to amines of the formula (III) in which R⁵ is an H, methyl, ethyl, n-propyl, isopropyl, n-butyl, 2-butyl, isobutyl, pentyl, hexyl, phenyl, in particular an H, ethyl, n-propyl, isopropyl, n-butyl, 2-butyl, isobutyl or phenyl, or a combination of the substituents mentioned.

The cyclic phosphonic anhydride can be added to the reaction medium either as a melt or as a liquid mixture dissolved in a solvent.

Suitable solvents are those which do not give rise to any side reactions with the phosphonic anhydride; these are all aprotic organic solvents, for example ligroin, butane, pentane, hexane, heptane, octane, cyclobutane, cyclopentane, cyclohexane, cycloheptane, cyclooctane, dichloromethane, chloroform, carbon tetrachloride, 1,2-dichloroethane, 1,1,2,2-tetrachloroethane, methyl acetate, ethyl acetate, propyl acetate, butyl acetate, dimethylformamide, diethylformamide, dimethylacetamide, diethylacetamide, diethyl ether, diisopropyl ether, tert-butyl methyl ether, THF, dioxane, acetonitrile or mixtures thereof; particular preference is given to dichloromethane, chloroform, ethyl acetate, propyl acetate, butyl acetate, dimethylformamide, diethylformamide, dimethylacetamide, diethylacetamide, diisopropyl ether, tert-butyl methyl ether, THF, dioxane, acetonitrile or mixtures thereof; very particular preference is given to dichloromethane, chloroform, ethyl acetate, butyl acetate, dimethylacetamide, tert-butyl methyl ether, THF, dioxane, acetonitrile or mixtures thereof; especially preferred are THF, ethyl acetate or butyl acetate.

The phosphonic anhydride is added generally in at least one third of the stoichiometric amount in relation to the starting compound, but may also be added in a superstoichiometric amount, for example in a ratio of 1 starting compound:1.2 T3P® (cyclic propanephosphonic anhydride).

The reactions are preferably carried out in such a way that the corresponding starting compound is added in a suitable solvent to T3P® at the reaction temperature.

The reaction product is preferably isolated by hydrolysis and simple phase separation, since the conversion products of the phosphonic anhydrides are generally very water-soluble. Depending on the nature of the product to be isolated, post-extractions may also be required. The phosphonic anhydride conversion product formed often does not disrupt subsequent reactions, so that even the direct use of the resulting reaction solutions often brings very good results. All procedures mentioned are notable for very good yields (typically 90-100%, in particular >95%) in the simultaneous absence of side reactions and epimerizations. The selectivities of the inventive reaction are in the range of 97-100%, in particular 99-100%.

The process according to the invention will be illustrated in detail by the examples which follow without restricting the invention thereto:

### EXAMPLE 1

### Elimination of 1-Phenylethanol to Styrene

1 mol of T3P® in ethyl acetate (50% w/w) is cooled to 0° C. 1-Phenylethanol is added dropwise to this solution and the mixture is stirred at this reaction for 3 hours. At this time, the reaction GC showed a conversion of 100%. After warming to room temperature, 180 ml of water were added and the phases were separated. After the solvent had been condensed off, the styrene remained in a yield of 97%, HPLC purity 99 % (a/a).

### EXAMPLE 2

### Elimination of 1-octanol to 1-octene

1 mol of T3P® in ethyl acetate (50% w/w) is cooled to 0° C. 1-Octanol is added dropwise to this solution and the mixture is stirred at this temperature for 3 hours. At this time, the reaction GC showed a conversion of 100%. After warming to room temperature, 180 ml of water were added and the phases were separated. After the solvent had been condensed off, the 1-octene remained in a yield of 97%, GC purity 99% (a/a).

### EXAMPLE 3

### Elimination of 1-cyclopentylprop-2-en-1-ol to allylidenecyclopentane

1 mol of T3P® in ethyl acetate (50% w/w) is cooled to 0° C. 1-Cyclopentylprop-2-en-1-ol is added dropwise to this solution and the mixture is stirred at this temperature for 3 hours. At this time, the reaction GC showed a conversion of 100%. After warming to room temperature, 180 ml of water were added and the phases were separated. After the solvent had been condensed off, the allylidenecyclopentane remained in a yield of 97%, GC purity 99% (a/a).

### EXAMPLE 4

### Elimination of (4R)-1-(tert-butoxycarbonyl)-4-hydroxy-L-proline methyl ester to 2,5-dihydropyrrole-1,2-(2S)-dicarboxylic acid 1-tert-butyl Ester 2-methyl ester

1 mol of T3P® in ethyl acetate (50% w/w) is cooled to 0° C. (4R)-1-(tert-butoxycarbonyl)-4-hydroxy-L-proline methyl ester is added dropwise to this solution and the mixture is stirred at this temperature for 3 hours. At this time, the reaction GC showed a conversion of 100%. After warming to room temperature, 180 ml of water were added and the phases were separated. After the solvent had been condensed off, the 2,5-dihydropyrrole-1,2-(2S)-dicarboxylic acid 1-tert-butyl ester 2-methyl ester remained in a yield of 97%, GC purity 99% (a/a).

1. A process for preparing alkenes of the formula (II)
R¹R²C═CR³R⁴ (II)
said process comprising
reacting
a) primary alcohols (R¹R²CHCH₂—OH) or
b) secondary alcohols (R¹R²CHCHR³—OH) or
c) tertiary alcohols (R¹R²CH—CR³R⁴OH)
with cyclic alkylphosphonic anhydrides at a reaction temperature in the range from −100 to +120° C.,
where R and/or R¹ and/or R² and/or R³ and/or R⁴ is H, a linear or branched C₁-C₁₂-alkyl radical, a C₃-C₁₀-cycloalkyl radical, alkenyl radical or an aryl or heteroaryl radical.

2. The process as claimed in claim 1, wherein the cyclic alkylphosphonic anhydride is a 2,4,6-substituted 1,3,5,2,4,6-trioxatriphosphinane 2,4,6-trioxide of the formula (I) 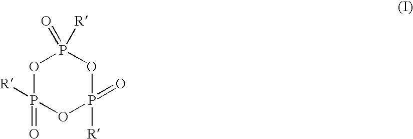
where R′ is independently allyl, aryl or open-chain or branched C₁ to C₁₂-alkyl radicals.

3. The process as claimed in claim 2, wherein R′ is selected from one or more of a methyl, ethyl, n-propyl, isopropyl, n-butyl, 2-butyl, isobutyl, pentyl, or hexyl, radical.

4. The process as claimed in claim 1, wherein the reaction is performed in the presence of an amine base of the formula (III)
NR⁵₃ (III)
where R⁵ is H, allyl, aryl or open-chain, cyclic or branched C₁ to C₁₂-alkyl radicals, aryloxy, allyloxy or alkoxy having open-chain cyclic or branched C₁ to C₁₂-alkyl radicals, or a combination thereof.

5. The process as claimed in claim 1, wherein the cyclic alkylphosphonic anhydride is added to the reaction solution either as a melt or dissolved in a solvent.

6. The process as claimed in claim 5, wherein the cyclic alkylphosphonic anhydride is added in a solvent and the solvent is aprotic.

7. The process as claimed in claim 1, wherein the primary secondary or tertiary alcohol is heated to the reaction temperature before the cyclic alkylphosphonic anhydride is added.

8. The process as claimed in claim 1, wherein the cyclic alkylphosphonic anhydride is used in one third of the stoichiometric amount up to a superstoichiometric amount in relation to the primary, secondary or tertiary alcohol.

9. The process as claimed in claim 2, wherein R′ is selected from one or more of an ethyl, propyl or butyl radical.
